(19) United States
(12) Patent Application Publication
(10) Pub. No.: US 2007/0244548 A1
(43) Pub. Date: Oct. 18, 2007

(54) Sugar-and drug-coated medical device

(75) Inventors: Randy Joe Myers, Bloomington, IN (US); Darin G. Schaeffer, Bloomington, IN (US); Stephanie Del Paine, West Lafayette, IN (US)

Correspondence Address: BRINKS HOFER GILSON & LIONE/CHICAGO/COOK, PO BOX 10395, CHICAGO, IL, 60610, US

(73) Assignees: Cook Incorporated, Bloomington, IN (US); MED Institute, Inc., West Lafayette, IN (US)

(21) Appl. No.: 11/709,375

(22) Filed: Feb. 22, 2007

Related U.S. Application Data
(60) Provisional application No. 60/777,157, filed on Feb. 27, 2006.

(51) Int. Cl. A61F 2/02

(52) U.S. Cl. 623/1.42

(57) ABSTRACT

The present invention relates to a medical device having a surface with a uniform or non-uniform layer posited thereon that includes a mono- or disaccharide sugar and at least one therapeutic agent. The present invention further relates to materials and a method of making such a medical device and methods of delivering a therapeutic agent.

### RELATED APPLICATIONS

This application claims benefit of provisional U.S. Patent Application Ser. No. 60/777,157, filed Feb. 27, 2006, which is incorporated herein by reference

### TECHNICAL FIELD

The present invention relates to the field of medicine with respect to medical devices designed for insertion into a patient's body.

### BACKGROUND

A variety of medical conditions are today treated, at least in part, by inserting a medical device into the body of an afflicted patient. For example, a stent may be used to prevent vessel occlusion, in one application, or to maintain the position of a graft used to repair a tissue within the body. A catheter can be employed for the purpose of transporting a graft or stent or both to a treatment site where there is a damaged tissue. Some medical treatments entail mechanically effecting tissue repair and/or excision by means of appropriately-equipped catheters and the like.

Medical devices may be inserted into the body temporarily or left in the body for extended periods, even indefinitely. For example, a stent may be implanted indefinitely within a body vessel to maintain vessel integrity, e.g., blood flow. These devices can be introduced, for example, into the esophagus, trachea, colon, biliary tract, urinary tract, vascular system or other location of a human or animal patient. For example, many treatments of the vascular system entail the introduction of a device such as a stent, catheter, balloon, wire guide, cannula, or the like, including combinations of such devices. When such devices are so used, however, body vessel walls may become damaged, possibly resulting in thrombosis and even stenosis.

Preferably, to mitigate any deleterious side effects such as thrombosis formation and stenosis, for example, medical devices can contain or be coated with a therapeutic agent that has a biological effect, which can be emitted from the device in a defined fashion. Medical devices that are coated with a therapeutic agent, at least in part, have been previously described in the art. However, many useful therapeutic agents have low solubility characteristics with respect to physiologically-acceptable solvents. Manufacture of a medical device having a sufficiently effective concentration of a useful therapeutic agent is thus difficult, time consuming, and often requires application of multiple layers of the same reagent to load the device sufficiently. Additionally, coating of therapeutic agents onto a medical device may result in a product that is not sufficiently durable and/or coated non-uniformly. Further issues may include insufficiently durable coatings and inconsistent dissolution of the therapeutic agent from the medical device.

### SUMMARY

In one embodiment of the present invention, a medical device comprising an implantable structure having a surface is provided. At least one layer is posited on at least a portion of the surface. The at least one layer comprises a mono- or disaccharide sugar and at least one therapeutic agent.

Preferably, the sugar component is present in an amount that is from about 10% to about 25% of the weight of the therapeutic agent. Preferred sugars used in the context of this embodiment are selected from the group consisting of galactose, glucose, mannose, xylose, sucrose, and trehalose. More preferred sugars include d-galactose, d-glucose, d-mannose, d-xylose, sucrose, and trehalose.

The present invention contemplated herein preferably pertains to an implantable structure selected from the group consisting of a stent, a wire guide, a catheter, a monitor, a prosthesis, a cannula, a graft, a cardiac pacemaker lead, a cardiac defibrillator lead, a suture, a needle, an angioplasty device, a pacemaker, an orthopedic device, appliance, implant or replacement, and a portion of any of these. Preferably, the implantable structure comprises a material selected from the group consisting of a biocompatible metal, carbon or carbon fiber, a biocompatible polymer, a biodegradable polymer, and an extracellular matrix component; wherein the biocompatible metal is selected from the group consisting of stainless steel, tantalum, titanium, nitinol, gold, platinum, inconel, iridium, silver, and tungsten, or alloys of any of these.

The therapeutic agent used in the context of the present invention is preferably selected from the group consisting of an anti-inflammatory agent, an analgesic agent, a local anesthetic agent, a vasospasm-inhibiting agent, a thrombolytic agent, an antithrombogenic agent, an antiproliferative agent, a fibrinolytic agent, a vasodilating agent, an antihypertensive agent, an antimicrobial agent, an antifungal agent, an antisecretory agent, an immunosuppressive agent, a dopamine agonist, a radiotherapeutic agent, a biological agent, an angiotensin converting enzyme (ACE) inhibitor, an antioxidant, a free radical scavenger, and an iron chelator or radiolabelled forms thereof or mixtures of two or more of these. More preferably, the therapeutic agent is an antiproliferative agent; wherein the antiproliferative agent inhibits microtubule disassembly or mitosis, for example. And most preferably, the antiproliferative agent is selected from the group consisting of paclitaxel, docetaxel, epothilone A, epothilone B, epothilone C, epothilone D, epothilone E, epothilone F, ixabepilone, camptothecin, colchicine, topotecan, vinblastine, vincristine, and vindesine, and analogs thereof.

Another preferred embodiment of the present invention relates to a method of delivering a therapeutic agent. The method comprises implanting a medical device comprising a surface and at least one layer posited on at least a portion of the surface. The layer comprises at least one mono- or disaccharide sugar and at least one therapeutic agent.

A further preferred embodiment of the present invention relates to a method of positing a therapeutic agent on a medical device. The method comprises forming a composition of the therapeutic agent and a mono- or disaccharide sugar, and contacting at least a part of the medical device with the composition for a time sufficient to allow the composition to adhere to the medical device.

### BRIEF DESCRIPTION OF THE DRAWINGS

FIG. 1 is a cross-sectional view of a medical device according to one embodiment of the invention.

FIG. 2 is a cross-sectional view of a medical device according to another embodiment of the invention.

FIG. 3 is a cross-sectional view of a medical device according to yet another embodiment of the invention.

FIG. 4 is a cross-sectional view of a medical device according to a further embodiment of the invention.

FIG. 5 is a cross-sectional view of a medical device according to an additional embodiment of the invention.

### DETAILED DESCRIPTION

The present invention relates to a medical device that is or includes an implantable structure. The implantable structure includes at least one layer of a coating adherent to it. The coating includes a sugar and a therapeutic agent. Sugars usefully employed for the present invention include any mono- or disaccharide. More particularly, the present invention allows for the coating of a sufficient quantity of a therapeutic agent onto the medical device so that the device meets certain standards of appearance and functionality. This process is particularly useful where the quantity is limited by the solubility characteristics of the therapeutic agent, and/or where the therapeutic agent layer is impaired with respect to lack of uniformity or smoothness of deposition, or results in uneven rates of dissolution therefrom.

In addition to the coated medical device, the present invention provides a method of making the coated medical device such that the absolute amount of the therapeutic agent, the proportioned amount of the therapeutic agent relative to the sugar, and the appearance of the coated medical device are each potentiated as compared to currently available methods.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Definitions

As used herein, the term “body vessel” means any tube-shaped body passage lumen that conducts fluid, including but not limited to blood vessels such as those of the human vasculature system, esophageal, intestinal, billiary, urethral and ureteral passages.

The term “biocompatible” refers to a material that is substantially non-toxic in the in vivo environment of its intended use, and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause a significantly adverse, long-lived or escalating biological reaction or response, and is distinguished from a mild, transient inflammation which typically accompanies surgery or implantation of foreign objects into a living organism.

The term “implantable” refers to an ability of a medical device to be positioned, for any duration of time, at a location within a body, such as within a body vessel. Furthermore, the terms “implantation” and “implanted” refer to the positioning, for any duration of time, of a medical device at a location within a body, such as within a body vessel.

As used herein, the phrase “therapeutic agent” refers to any pharmaceutically active agent that results in an intended therapeutic effect on the body to treat or prevent conditions or diseases. Therapeutic agents include any suitable biologically-active chemical compounds, biologically derived components such as cells, peptides, antibodies, and polynucleotides, and radiochemical therapeutic agents, such as radioisotopes.

The term “coating,” as used herein and unless otherwise indicated, refers generally to material attached to an implantable medical device prior to implantation. A coating can include material covering any portion of a medical device, and can include one or more coating layers. A coating can have a substantially constant or a varied thickness and composition. Coatings can be adhered to any portion of a medical device surface, including the luminal surface, the abluminal surface, or any portions or combinations thereof.

The term “pharmaceutically acceptable,” as used herein, refers to those compounds of the present invention which are, within the scope of sound medical judgment, suitable for use in contact with the tissues of humans and lower mammals without undue toxicity, irritation, and allergic response, are commensurate with a reasonable benefit/risk ratio, and are effective for their intended use, as well as the zwitterionic forms, where possible, of the compounds of the invention.

By “pharmaceutically acceptable salt” is meant those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharm Sciences,66: 1-19 (1977), which is hereby incorporated by reference.

The term “medical device” means any object that is itself or that includes a component that is intentionally inserted into the body of a patient as part of a medical treatment, and that comprises a structure adapted for introduction into a patient. The medical device can be a tool, such as, without limitation, a catheter, a wire guide, a foreceps, or a scissors used to effect a surgical procedure at and/or deliver a second medical device to a treatment site in a patient. Alternatively, the medical device can be a temporary implant that imparts an electrical pulse as in a pacemaker or a chemical pulse (or aliquot) as in a drug-delivery instrument, to name two examples. Yet another alternative medical device of the present invention is one that is commonly intended to be a permanent implant, such as, for example, a graft or a stent.

Accordingly, the medical device of the present invention includes or is an implantable structure that is, without limitation, preferably selected from the group consisting of a stent, a wire guide, a catheter, a monitor, a prosthesis, a cannula, a graft, a cardiac pacemaker lead, a cardiac defibrillator lead, a suture, a needle, an angioplasty device, a pacemaker, an orthopedic device, appliance, implant or replacement, a scalpel, a scissors, and a forceps, and a portion of any of these. Particularly preferred prostheses include valves, such as, without limitation, heart valves or venous valves, wherein the valve is preferably formed of a biological valve, a biologically-derived material that did not form a valve in vivo, or a synthetic material, such as a nylon or some other polymer. The biologically-derived material preferably includes at least one of the following: a protein, an extracellular matrix component, a collagen, a fibrin, and mixtures of any of these. The catheter usefully employed in the context of this invention can include one or more inflatable members, as in a balloon catheter, or not. The medical device can include one or more concavities, such as, without limitation, wells or grooves, where additional solution containing a bioactive agent can accumulate in the layering process. Despite the naming of exemplary medical devices as set forth above, the set of medical devices usefully employed in the context of the present invention should not be so limited.

The Medical Device

As noted above, the medical device of the present invention is any object that is purposefully inserted into a patient's body as part of a medical treatment, irrespective whether the insertion is intended to be a temporary measure implemented with respect to a surgical procedure, such as may occur when a patient requires angioplasty with the insertion of an inflatable-member catheter, for example, or a temporary measure implemented with respect to measuring certain physiological characteristics, such as in assessment of internal sugar levels over time via an implantable monitor, or a permanent measure implemented with respect to repair of a tissue using a graft and/or a stent, for example. One example of a medical device of the present invention is processed extracellular matrix material per se that is isolated from an animal, as further described in U.S. Pat. No. 6,206,931 and 6,666,892, which are respectively incorporated herein by reference. Grafts and other medical devices that include such material are currently sold by Cook Inc. under the SIS trademark. As a further example, such material can also be used in the manufacture of a prosthesis, such as, for example, a valve.

Accordingly, one embodiment of a medical device of the present invention that includes an implantable structure is depicted with respect to a segment of the medical device, as represented by the drawings that are framed by zigzag lines at the top and bottom of each of FIGS. 1-5. The zigzag lines signify that the device continues beyond the location of the zigzag lines, i.e., only a portion of the structure 12 is shown in each of the Figures.

By way of example, the structure 12 depicted in the Figures can be configured as a vascular stent particularly adapted for insertion into the vascular system of a patient. Of course, such a stent structure can be used in other systems and sites, such as the esophagus, trachea, colon, biliary ducts, urethra and ureters, among others. Indeed, the structure 12 can alternatively be configured as any conventional vascular or other medical device, and can include any of a variety of conventional stent or other adjuncts, such as helically wound strands, perforated cylinders, or the like.

Moreover, the structure 12 need not represent the entire device, but can merely be that portion of a medical device that includes a suitable coating on its surface. A suitable coating is one that includes at least one layer that comprises a therapeutic agent and a sugar, as further described herein, which therapeutic agent is intended to be imparted into the patient. The coating can also include one or more layers that are substantially devoid of the therapeutic agent. For example, a priming layer can be employed to cover the implantable structure to increase adherence of succeeding layers and/or to encase deleterious or toxic material that may be used to form the implantable structure. Another example is a protecting layer that overlays a therapeutic agent layer to protect it from inadvertent touching or the atmosphere or to control or otherwise impact the rate of elution of the therapeutic agent.

The protecting layer can also be used to separate therapeutic agent layers, particularly when otherwise adjoining layers include different therapeutic agents that are preferably kept substantially separate, or substantially separate at least until the point and time of delivery to the site treatment. Succeeding therapeutic agent layers can include varying ratios of the therapeutic agent and sugar as a means to impact elution rate and localized concentration of the therapeutic agent. The layers may also be preferably separated by a protecting layer in another embodiment.

Accordingly, the structure 12 can be configured as at least one of, or any portion of, a catheter, a wire guide, a cannula, a stent, a vascular or other graft, a cardiac pacemaker lead or lead tip, a cardiac defibrillator lead or lead tip, a heart valve, a suture, a needle, an angioplasty device, a pacemaker or an orthopedic device, appliance, implant or replacement. The structure 12 can also be configured as a combination of these various devices or portions of any of these. Any device can include concavities, such as wells or grooves, among others, where additional quantities of therapeutic agent can be deposited relative to non-concave regions of the device surface.

Most preferably, however, the structure 12 is configured as a vascular stent, such as the commercially-available Gianturco-Roubin FLEX-STENT coronary stent from Cook Incorporated, Bloomington, IN. Such stents are typically about 10 mm to about 60 mm in length and designed to expand to a diameter of about 2 mm to about 6 mm when inserted into the vascular system of the patient. The Gianturco-Roubin stent, in particular, is typically about 12 mm to about 25 mm in length and designed to expand to a diameter of about 2 mm to about 4 mm when so inserted.

These stent dimensions are applicable to exemplary stents employed in the coronary arteries. Structures such as stents or catheter portions intended to be employed at other sites in a patient, such as in the aorta, esophagus, trachea, colon, biliary tract or urinary tract, will have different dimensions more suited to such use. For example, aortic, esophageal, tracheal and colonic stents may have diameters up to about 25 mm and lengths of about 100 mm or longer.

The structure 12 is composed of a material 14 suitable for the intended use of the structure 12, which material 14 is metal, plastic, or organic matter (e.g., extra-cellular matrix, chitin, and cellulose, among others), to name a few alternatives. The material 14 is preferably biocompatible, although cytotoxic or other poisonous materials may be employed if they are adequately isolated from the patient's tissues, or purposefully exposed to tissue at the treatment site. Such incompatible materials may be useful in, for example, radiation treatments in which a radioactive material is positioned by a medical device designed for the function, such as a suitably designed catheter, in or close to the specific tissues to be treated. Under most circumstances, however, the material 14 of the structure 12 is preferably biocompatible, and preferably comprises, for example, a surgical grade metal (such as stainless steel) or a biodegradable polymer prepared from natural sources or synthetically, such as an extracellular matrix component or components, poly(lactic acid), and the like, as more fully set forth below in the section titled “Alternative Structure Materials.”

Referring to FIG. 1, an embodiment of a medical device 10 in accordance with the present invention is represented. The medical device comprises the structure 12 adapted for introduction into a human or animal patient. The modifier “adapted” means that the structure 12 is shaped and sized for such introduction. The therapeutic agent having suitable bioactivity is included in layer 18, which, in this embodiment, is deposited in direct contact with the material 14 of the structure 12. There can be one or more different therapeutic agents included in the therapeutic agent layer 18. For direct deposition of layer 18 onto the material 14, it may be advantageous to process or activate the surface of the material 14 so that the applied therapeutic agent of layer 18 more efficiently adheres to the material 14. It may also be advantageous to include concavities in the structure, such as wells or grooves, where additional solution for applying the therapeutic agent can collect. The resulting coated medical device will have an increased amount of therapeutic agent adhering at such concave sites, which may or may not be concave after the coating is applied. Assuring adhesion of the therapeutic agent layer 18 is especially important, whether it is the only layer added to the medical device 10, or an optional protecting layer 20 is added on top as shown. The protecting layer 20 is preferably included, in some embodiments of the present invention, when the rate of delivery of the therapeutic agent of layer 18 into the patient's body is usefully further controlled over the rate of delivery of the therapeutic agent that is provided by the layer 18 alone. Surface processing and surface activation can also selectively alter the release rate of the therapeutic agent. Accordingly, contemplated embodiments of the present invention include medical devices that have the therapeutic agent layer 18 attached directly to the structure 12, with or without a protecting layer 20 that comprises, in one embodiment, a porous material that impacts rate of elution of the therapeutic agent from the medical device when inserted into a patient.

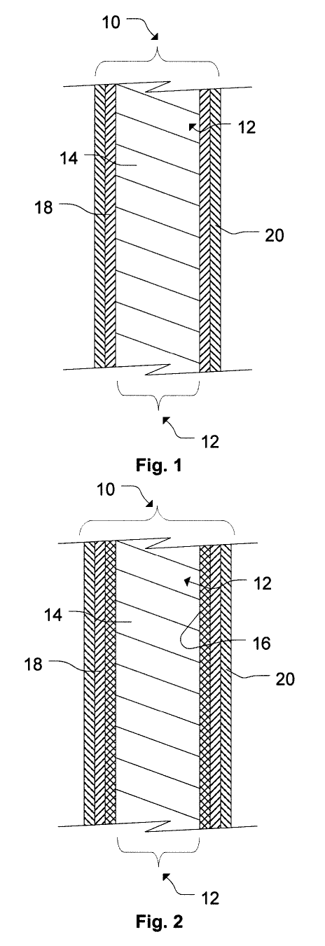

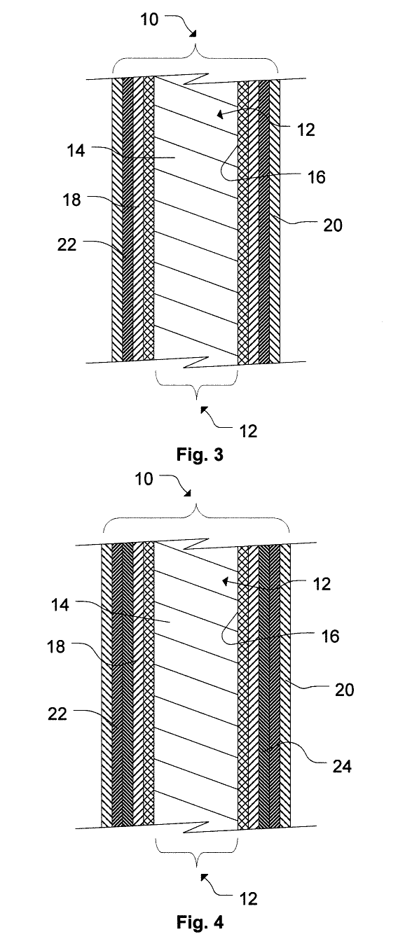

Alternative embodiments of the medical device 10 are depicted in FIGS. 2-5. FIG. 2 shows the inclusion of a primer layer 16 that is deposited directly on the material 14 in certain embodiments, the purpose of which is to mask deleterious characteristics of the material 14, if any exist, and/or to provide a surface that is more conducive to adherence of layer 18, and/or to impact the rate of elution of the therapeutic agent. FIG. 3 shows the embodiment where a second therapeutic agent layer 22 is included, which, like layer 18, includes at least one therapeutic agent. The present invention includes the embodiment where therapeutic agent layers are added sequentially without any separation of these layers, as shown in FIG. 3, as well as the embodiment where a second protecting layer 24 separates the two therapeutic agent layers 18 and 22, as shown in FIG. 4. The protecting layers provide control of the dissolution of the therapeutic agent as well as contribute to the durability of the medical device 10 itself.

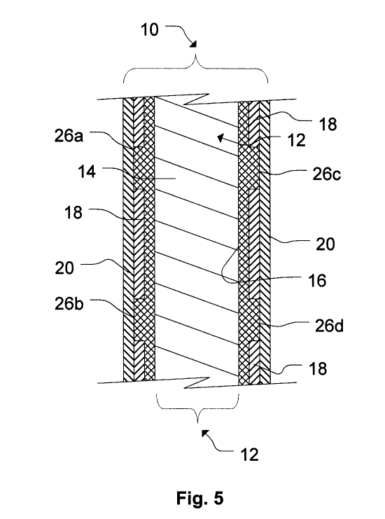

FIG. 5 shows an embodiment of the medical device 10 in which a mechanical bond or connector 26 is provided between (a) any one of the protecting layers 20 and 24, and (b) any or all of the other of the protecting layers 20 and 24, the primer layer 16 and the material 14. The connector 26 reliably secures the layers 16, 20 and/or 24 to each other, and/or to the material 14. The connector 26 lends structural integrity to the medical device 10, particularly after the therapeutic agent layer or layers 18 and/or 22 have been fully released into the patient.

It should be noted that the therapeutic agent in the present invention is delivered by elution from the surface of the medical device 10, and not by passage from an outside source, such as through any lumen that may be present in the medical device 10. For example, the present invention is distinct from a catheter employed in conventional chemotherapy, where a cancer therapeutic drug is delivered only via an internal lumen. Such a device could additionally employ the drug-delivery approach of the present invention. For example, for those applications where the therapeutic agent is combined with a second therapeutic agent, the second therapeutic agent can be introduced from an outside source via a lumen in the medical device. The therapeutic agent of the present invention may, of course, be released from the medical device 10 into the lumen defined in the device. Preferably, the therapeutic agent is delivered to tissue in contact with the device at a location where the second agent is deposited via a lumen from the outside. Accordingly, the lumen of the medical device 10 may deliver one or more therapeutic agents, which may include the therapeutic agent that is imparted from the surface of the medical device 10.

A vast range of drugs can be employed as the therapeutic agent included in the layer 18. In addition to having useful bioactivity for internally treating a patient, a suitable therapeutic agent preferably survives exposure to a vacuum drawn during vapor deposition or plasma deposition on the medical device. Particularly useful in the practice of the present invention are materials that prevent or ameliorate abrupt closure and restenosis of blood vessels previously opened by stenting surgery or other procedures. Another category of highly preferred therapeutic agents used in the context of the present invention is the antiproliferatives. Particularly preferred antiproliferatives are those that inhibit microtubule disassembly, a vital cellular activity of actively dividing cells of a cancer. Included in this class are taxoid and epothilone compounds. Preferred taxoid compounds include docetaxel and paclitaxel. Preferred epothilone compounds include epothilone B and ixabepilone. Another group of preferred antiproliferatives are those that inhibit mitosis, such as alkyloid compounds. These and other categories and species of therapeutic agents are set forth in further detail below in the subsection labeled “Alternative Therapeutic Agents.”

The present invention provides a method for potentiating the appearance, uniformity of deposition, durability, quantity, and consistency of dissolution of the therapeutic agent that is deposited as layer 18 and/or layer 22 of a medical device 10, which method includes a composition of the therapeutic agent in the presence of a suitable sugar. The composition preferably also includes a suitable solvent. The suitable solvent is one that has a sufficient degree of volatility so that when sprayed onto a medical device or otherwise applied thereto, the solvent evolves away within a 60-minute period, more preferably within a 30-minute period, yet more preferably within a 10-minute period, even more preferably within one minute; wherein the evolution of the solvent preferably occurs at ambient temperature and pressure, more preferably occurs at a temperature that is between ambient temperature and up to about five degrees below the temperature at which the therapeutic agent denatures or otherwise destabilizes, and the pressure is preferably left at ambient levels or reduced. A most preferred set of conditions under which the evolution of solvent occurs is at substantially reduced pressure, as within a chamber attached to an aspirator or vacuum pump, and at ambient temperature; another most preferred set of conditions is under the reduced pressure just noted coupled to elevated temperature up to no more than about five degrees below the point of heat denaturation of the therapeutic agent.

Suitable solvents in accordance with the present invention include, without limitation, anhydrous alcohol, including, for example, ethanol or methanol; water-diluted alcohol, for example, a water-ethanol or water-methanol mixture; dimethylsulfoxide (DMSO); and acetonitrile (ACN). A common feature of the suitable solvents is the approximate volatility of each of them, which is preferably approximately that of anhydrous ethanol, plus or minus 20% thereof; more preferably, plus or minus 15% thereof; yet more preferably, plus or minus 10% thereof. Preferred solvents used in accordance with the present invention include methanol and DMSO, and mixtures thereof.

Suitable solvents are also defined in part by their ability to be at least partially miscible with the therapeutic agent of interest. Obviously, different therapeutic agents have differing capabilities to dissolve in different solvents; of course, some will not dissolve in a given solvent. Alternatively, a therapeutic agent may form suspensions in some liquids, which suspensions are included in the use of the term “composition” used herein. Preferably, a liquid approach to uniform layering of the therapeutic agent is contemplated in the present inventive method. A composition that contains the therapeutic agent is preferably a liquid solution or mixture that includes the sugar component as well. The therapeutic agent can be fully or partially solubilized, or suspended (i.e., not dissolved), in the composition. To the extent that the therapeutic agent does not dissolve in a solvent, the solvent remains suitable to the extent that the solvent plus therapeutic agent of choice forms a stable suspension in the presence of the sugar whereby a uniform layering of therapeutic agent can be applied to the medical device.

One theory of the function of sugar in the present invention is that it serves as a cosolvent, thereby increasing the solubility of a substance that has limited solubility in water (as in the case of taxoid compounds, for example). Irrespective whether this theory is correct or not, the combination of the sugar and a therapeutic agent provides characteristics that increase the uniform application and reduce the friability or brittleness of the therapeutic agent on the medical device upon drying. Suitable sugars will not evolve upon drying of the medical device after application of the solution containing the therapeutic agent plus sugar. Instead, the preferred sugar will remain with the therapeutic agent attached to the medical device, forming a smooth layer. Moreover, whereas the inclusion of sugar presents the advantage of maximizing the concentration of the therapeutic agent in a composition used for applying the therapeutic agent layer onto the medical device, that characteristic also presents the advantage of providing a broader range of concentrations that may be manipulated and over which the therapeutic agent can be controllably layered onto the medical device.

These characteristics are associated with a consistent elution of the therapeutic agent from the medical device, and with at most insignificant deleterious interaction between the therapeutic agent and the medical device itself. Suitable sugars have insignificant toxic or allergic effects in the concentrations contemplated herein on human or animal patients. The concentration of sugar in the composition that is applied to the medical device ranges from about 0.001 M to about 1 M; preferably, the concentration of sugar is about 0.005 M, about 0.01 M, about 0.033 M, about 0.067 M, about 0.1 M, about 0.133 M, about 0.167 M, about 0.2 M, about 0.233 M, about 0.267 M, about 0.3 M, about 0.333 M, about 0.367 M. about 0.4 M, about 0.433 M, about 0.467 M, about 0.5 M, about 0.55 M, about 0.6 M, about 0.65 M, about 0.7 M, about 0.75 M, about 0.8 M, about 0.85 M, about 0.9 M, about 0.95 M, about 1 M. Yet greater concentrations of sugar included in the composition are contemplated as well, such as, for example, about 1.33 M, about 1.67 M, about 2 M, and above.

Suitable sugars include mono- or disaccharides that, with respect to monosaccharide sugars, are composed of from about two to about seven carbon atoms, and, with respect to disaccharide sugars, from about four to about 14 carbon atoms. One of the carbons carries an aldehydic or ketonic oxygen, which may be combined in acetal or ketal forms. The remaining carbons usually have hydrogen atoms and hydroxyl groups. Preferred monosaccharide sugars usefully employed in the context of the present invention include, without limitation: three-carbon species, such as glyceraldehydes and dihydroxyacetone; four-carbon species, such as erythrose, threose, erythrulose; five-carbon species, such as ribose, arabinose, xylose, lyxose, ribulose, xylulose; six-carbon species, such as allose, altrose, glucose, mannose, gulose, idose, galactose, talose, psicose, fructose, sorbose, tagatose; and seven-carbon species. Preferred disaccharide sugars usefully employed in the context of the present invention include any pair combination of two of the more preferred monosaccharide sugar species, including pairs of the same preferred monosaccharide sugar species, such as but not limited to sucrose, lactose, maltose, trehalose, and cellobiose.

For the purposes of the present invention, all configurational isomeric forms of each of the monosaccharide sugars can be employed, including, without limitation stereoisomers, diastereoisomers, epimers, anomers, optical isomers, enantiomers, and the like. Moreover, either or both open chain forms as well as cyclized ring forms thereof may also be employed in the present invention.

Of the preferred mono- and disaccharides identified, more preferred such sugars include galactose, glucose, mannose, xylose, fructose, sucrose, and trehalose. Alternate monosaccharide sugar species include any of d-galactose, d-glucose, d-mannose, and d-xylose. The most preferred monosaccharide sugar species is d-glucose. Preferred disaccharide sugar species are sucrose, lactose, trehalose, and maltose. The most preferred disaccharide sugar species is sucrose.

The sugar as used in the context of the present invention is included in the solution in the range preferably of from about 10% to about 25% (w/w) with respect to the weight of therapeutic agent to be added to the composition. More preferably, the range of weight ratio is from about 12% to about 22%. Yet more preferably, the range of weight ratio is from about 15% to about 20%.

The amount of therapeutic agent used is determined by adding the therapeutic agent to a given volume of solvent until the composition is saturated. Preferably, the solvent is heated to within about five degrees of the temperature at which the therapeutic agent denatures or degrades. Once the saturation level is determined, a mass of sugar is prepared that is between about 10% and about 25% of the weight of the saturating amount of therapeutic agent that was added in the aforementioned determination of the saturating level of therapeutic agent in the solvent of choice. Keeping the starting volumes the same, now using a liquid formed of the combination of the original solvent plus the sugar, one adds the therapeutic agent of choice to the point of saturation, preferably increasing the temperature of the liquid to no greater than about five degrees below the point of denaturing/degrading temperature of the therapeutic agent. This solution is used in the method further set forth herein for applying a layer or layers of therapeutic agent to the implantable structure of the medical device.

This composition comprising solvent, sugar, and therapeutic agent is especially useful in applying therapeutic agents that have high degrees of hydrophobicity. Exemplary such therapeutic agents include, without limitation, docetaxel and paclitaxel. The amounts of therapeutic agent per volume of solvent-sugar mixtures, uniformity of application thereof, and appearance after drying on medical devices of the present invention are determinations and procedures that are within the skill set of the ordinary artisan. In particular, sugar solutions ranging from about 0.001 M to about 1 M concentration, using intermediate concentrations in steps of about 0.005 to about 0.05 increments are usefully employed in finding a useful range of sugar concentration for dissolving the therapeutic agent for application to the medical device of the present invention.

For example, one can select a range of concentrations, apply them onto a medical device, and review the effect on a control protecting layer to which sugar has not been added. If a marked reduction in the quantity of crystalline artifacts found per unit surface area of the device is observed, one can refine further the optimal concentration of sugar to include.

Generally, the sugar-containing layered coat appears smooth and glassy, and is notable for its general lack of crystalline artifacts as compared to the same coat in the absence of sugar.

Further description of the process of applying the solvent-sugar-therapeutic agent composition is set forth below as well, in the section titled “Description Regarding Medical Device Design and Coating.”

Alternative Structure Materials

A variety of conventional materials can be employed as the material 14. Some materials may be more useful for structures other than the coronary stent exemplifying the structure 12. The material 14 may be elastic or inelastic, depending upon the flexibility or elasticity of the layers of therapeutic agent(s) and other materials to be applied over it, and may be elastic in one part and inelastic in a second part of the structure 12, and thus may contain different materials at such different parts. The material may be biodegradable or nonbiodegradable. Moreover, the therapeutic agent itself may have sufficient strength to serve as the material 14 of some useful structures 12, even if not especially useful in the exemplary coronary stent. Overall, the structure 12 preferably comprises a material selected from the group consisting of a biocompatible metal, alloy, carbon-to-carbon fiber, a biocompatible polymer, a biodegradable polymer, and an extracellular matrix component, or combinations of these materials.

Accordingly, the material 14 can include at least one of the group of biocompatible metals or alloys consisting of stainless steel, tantalum, titanium, nitinol, gold, platinum, inconel, iridium, silver, tungsten, or alloys of any of these. An alternative form of the material 14 employs a carbon or carbon fiber material. Another material 14 employs a biocompatible polymer, such as one selected, without limitation, from the group consisting of cellulose acetate, cellulose nitrate, silicone, polyethylene teraphthalate, polyurethane, polyamide, polyester, polyorthoester, polyanhydride, polyether sulfone, polycarbonate, polypropylene, high molecular weight polyethylene, and polytetrafluoroethylene, or mixtures or copolymers of these.

Yet another alternative form of the material 14 employs a biodegradable polymer, such as one selected from the group consisting of polycaprolactone, polyhydroxybutyrate valerate, a poly(L-lactide) (PLLA), a poly lactic acid [poly(D,L-lactide) (PLA)], a polyglycolic acid [polyglycolide (PGA)], a poly(L-lactide-co-D,L-lactide) (PLLA/PLA), a poly(L-lactide-co-glycolide) (PLLA/PGA), a poly(D, L-lactide-co-glycolide) (PLA/PGA), a poly(glycolide-co-trimethylene carbonate) (PGA/PTMC), a poly(D,L-lactide-co-caprolactone) (PLA/PCL), a poly(glycolide-co-caprolacto- ne) (PGA/PCL); a polyethylene oxide (PEO), a polydioxanone (PDS), a polypropylene fumarate, a poly(ethyl glutamate-co-glutamic acid), a poly(tert-butyloxy-carbonylmethyl glutamate), a polycaprolactone (PCL), a polycaprolactone co-butylacrylate, a polyhydroxybutyrate (PHBT), a poly(phosphazene), a poly(phosphate ester), a poly(amino acid), a polydepsipeptide, a polyanhydride, such as a polymaleic anhydride, a polyiminocarbonate, a poly[(97.5% dimethyl-trimethylene carbonate)-co-(2.5% trimethylene carbonate)], a cyanoacrylate, a polyethylene oxide, a hydroxypropylmethylcellulose, a polysaccharide, and a protein, or mixtures or copolymers thereof. The polysaccharide used for the material 14 in the context of the present invention is preferably selected from the group consisting of hyaluronic acid, chitosan, and cellulose, or mixtures or copolymers thereof. The protein used for the material 14 in the context of the present invention is preferably selected from the group consisting of gelatin, collagen, and fibrin, or mixtures or copolymers thereof.

Stainless steel is particularly useful as the material 14 when the structure 12 is configured as a vascular stent.

When the structure 12 is composed of a radiolucent material, such as polypropylene, polyethylene or others noted above, a conventional radiopaque coating may and preferably should be applied to it. The radiopaque coating provides a means for identifying the location of the structure 12 by X-ray or fluoroscopy during or after its introduction into a patient's vascular system. Suitable radiopaque coatings preferably employed in the context of the present invention include one of more selected from the group consisting of a heavy metal element, an iodine-containing compound, and a barium-containing compound. Heavy elements that are usefully employed with the present invention include, without limitation, gold, platinum, tantalum, tungsten, and combinations thereof or alloys of one or more of these and a second element that is not a heavy element.

A preferred material or form or shape thereof used for the structure 12 has characteristics that render the medical device 10 substantially unaffected by the high magnetic field strengths used for magnetic resonance imaging, which can impact the temperature and the controlled release of the therapeutic agent of the coating. Such preferred materials include aluminum alloys, titanium, plastics, and nanometric films that neutralize or retard the effect of high magnetic fields.

Alternative Therapeutic Agents

As noted above, many different therapeutic agents have been identified as usefully employed in the context of the medical device of the present invention. More generally, the present invention can be employed with respect to a therapeutic agent selected, without limitation intended, from the group consisting of an anti-inflammatory agent, an analgesic agent, a local anesthetic agent, a vasospasm-inhibiting agent, a thrombolytic agent, an antithrombogenic agent, an antiproliferative agent, a fibrinolytic agent, a vasodilating agent, an antihypertensive agent, an antimicrobial agent, an antifungal agent, an antisecretory agent, an immunosuppressive agent, a dopamine agonist, a radiotherapeutic agent, a biological agent, an angiotensin converting enzyme (ACE) inhibitor, an antioxidant, a free radical scavenger, an iron chelator, or radiolabelled forms thereof, or mixtures of two or more of these.

A particularly useful therapeutic agent in the category of anti-inflammatory agent is one that is preferably non-steroidal and is selected, without limitation, from the group consisting of an arboxylic acid derivative, an arylacetic acid derivative, an arylbutyric acid derivative, an arylcarboxylic acid, an arylpropionic acid derivative, a pyrazole, a pyrazolone, a salicylic acid derivative, a thiazinecarboxamide, ε-acetamidocaproic acid, s-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap, or pharmaceutically acceptable salts thereof, or mixtures of two or more of these.

Of these, a preferred species of the arboxylic acid derivative is selected, without limitation, from the group consisting of enfenamic acid, etofenamate, flufenamic acid, isonixin, meclofenamic acid, mefanamic acid, niflumic acid, talniflumate, terofenamate, and tolfenamic acid, or mixtures of two or more of these. A preferred species of the arylacetic acid derivative is selected, without limitation, from the group consisting of an acemetacin, an alclofenac, an amfenac, a bufexamac, a cinmetacin, a clopirac, a diclofenac sodium, etodolac, felbinac, fenclofenac, fenclorac, fenclozic acid, fentiazac, glucametacin, ibufenac, indomethacin, isofezolac, isoxepac, lonazolac, metiazinic acid, oxametacine, proglumetacin, sulindac, tiaramide, tolmetin, and zomepirac, or mixtures of two or more of these. A preferred species of the arylbutyric acid derivative is selected, without limitation, from the group consisting of bumadizon, butibufen, fenbufen, and xenbucin, or mixtures of two or more of these. A preferred species of the arylcarboxylic acid is selected, without limitation, from the group consisting of clidanac, ketorolac, and tinoridine, or mixtures of two or more of these. A preferred species of the arylpropionic acid derivative is selected, without limitation, from the group consisting of alminoprofen, benoxaprofen, bucloxic acid, carprofen, fenoprofen, flunoxaprofen, flurbiprofen, ibuprofen, ibuproxam, indoprofen, ketoprofen, loxoprofen, miroprofen, naproxen, oxaprozin, piketoprofen, pirprofen, pranoprofen, protizinic acid, suprofen, tiaprofenic acid, and mixtures of two or more of these. A preferred species of the pyrazole is, without limitation, difenamizole or epirizole or a mixture of the two. A preferred species of the pyrazolone is selected, without limitation, from the group consisting of apazone, benzpiperylon, feprazone, mofebutazone, morazone, oxyphenbutazone, phenybutazone, pipebuzone, propyphenazone, ramifenazone, suxibuzone, and thiazolinobutazone, or mixtures of two or more of these. A preferred species of the salicylic acid derivative is selected, without limitation, from the group consisting of acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamine o-acetic acid, salicylsulfuric acid, salsalate, and sulfasalazine, or mixtures of two or more of these. A preferred species of the thiazinecarboxamide is selected, without limitation, from the group consisting of droxicam, isoxicam, piroxicam, and tenoxicam, or mixtures of two or more of these.

Another particularly useful therapeutic agent in the category of anti-inflammatory agent is one that is preferably steroidal and is selected, without limitation, from the group consisting of 21-acetoxyprefnenolone, alclometasone, algestone, amicinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, dexamethasone sodium phosphate, dexamethasone acetate, diflorasone, diflucortolone, difluprednate, enoxolone, fluazacort, flucloronide, flumehtasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol priopionate, halometasone, halopredone acetate, hydrocortamate, hydrocortisone, loteprednol etabonate, mazipredone, medrysone, meprednisone, methyolprednisolone, mometasone furoate, paramethasone, prednicarbate, prednisolone, prednisolone 25-diethylaminoacetate, prednisone sodium phosphate, prednisone, prednival, prednylidene, rimexolone, tixocortal, triamcinolone, triamcinolone acetonide, triamcinolone benetonide, and triamcinolone hexacetonide, or pharmaceutically acceptable salts thereof, or mixtures of two or more of these.

A particularly useful therapeutic agent in the category of analgesic agent is one that is preferably a narcotic compound that is selected, without limitation, from the group consisting of alfentanil, allylprodine, alphaprodine, anileridine, benzylmorphine, bezitramide, buprenorphine, butorphanol, clonitazene, codeine, codeine methyl bromide, codeine phosphate, codeine sulfate, desomorphine, dextromoramide, dezocine, diampromide, dihydrocodeine, dihydrocodeinone enol acetate, dihydromorphine, dimenoxadol, dimepheptanol, dimethylthiambutene, dioxaphetyl butyrate, dipipanone, eptazocine, ethoheptazine, ethylmethlythiambutene, ethylmorphine, etonitazene, fentanyl, hydrocodone, hydromorphone, hydroxypethidine, isomethadone, ketobemidone, levorphanol, lofentanil, meperidine, meptazinol, metazocine, methadone hydrochloride, metopon, morphine, myrophine, nalbuphine, narceine, nicomorphine, norlevorphanol, normethadone, normorphine, norpipanone, opium, oxycodone, oxymorphone, papaveretum, pentazocine, phenadoxone, phenazocine, pheoperidine, piminodine, piritramide, proheptazine, promedol, properidine, propiram, propoxyphene, rumifentanil, sufentanil, and tilidine, or pharmaceutically acceptable salts thereof, or mixtures of two or more of these.

Another particularly useful therapeutic agent in the category of analgesic agent is one that is preferably non-narcotic and is selected, without limitation, from the group consisting of aceclofenac, acetaminophen, acetaminosalol, acetanilide, acetylsalicylsalicylic acid, alclofenac, alminoprofen, aloxiprin, aluminum bis(acetylsalicylate), aminochlorthenoxazin, 2-amino-4-picoline, aminopropylon, aminopyrine, ammonium salicylate, amtolmetin guacil, antipyrine, antipyrine salicylate, antrafenine, apazone, aspirin, benorylate, benoxaprofen, benzpiperylon, benzydamine, bermoprofen, brofenac, p-bromoacetanilide, 5-bromosalicylic acid acetate, bucetin, bufexamac, bumadizon, butacetin, calcium acetylsalicylate, carbamazepine, carbiphene, carsalam, chloralantipyrine, chlorthenoxazin(e), choline salicylate, cinchophen, ciramadol, clometacin, cropropamide, crotethamide, dexoxadrol, difenamizole, diflunisal, dihydroxyaluminum acetylsalicylate, dipyrocetyl, dipyrone, emorfazone, enfenamic acid, epirizole, etersalate, ethenzamide, ethoxazene, etodolac, felbinac, fenoprofen, floctafenine, flufenamic acid, fluoresone, flupirtine, fluproquazone, flurbiprofen, fosfosal, gentisic acid, glafenine, ibufenac, imidazole salicylate, indomethacin, indoprofen, isofezolac, isoladol, isonixin, ketoprofen, ketorolac, p-lactophenetide, lefetamine, loxoprofen, lysine acetylsalicylate, magnesium acetylsalicylate, methotrimeprazine, metofoline, miroprofen, morazone, morpholine salicylate, naproxen, nefopam, nifenazone, 5′ nitro-2′ propoxyacetanilide, parsalmide, perisoxal, phenacetin, phenazopyridine hydrochloride, phenocoll, phenopyrazone, phenyl acetylsalicylate, phenyl salicylate, phenyramidol, pipebuzone, piperylone, prodilidine, propacetamol, propyphenazone, proxazole, quinine salicylate, ramifenazone, rimazolium metilsulfate, salacetamide, salicin, salicylamide, salicylamide o-acetic acid, salicylsulfuric acid, salsalte, salverine, simetride, sodium salicylate, sulfamipyrine, suprofen, talniflumate, tenoxicam, terofenamate, tetradrine, tinoridine, tolfenamic acid, tolpronine, tramadol, viminol, xenbucin, and zomepirac, or pharmaceutically acceptable salts thereof, or mixtures of two or more of these.

A particularly useful therapeutic agent in the category of anesthetic agent is one that is selected, without limitation, from the group consisting of amucaine, amolanone, amylocalne hydrochloride, benoxinate, benzocaine, betoxycaine, biphenamine, bupivacaine, butacaine, butaben, butanilicaine, butethamine, butoxycaine, carticaine, chloroprocaine hydrochloride, cocaethylene, cocaine, cyclomethycaine, dibucaine hydrochloride, dimethisoquin, dimethocaine, diperadon hydrochloride, dyclonine, ecgonidine, ecgonine, ethyl chloride, beta-eucaine, euprocin, fenalcomine, fomocaine, hexylcaine hydrochloride, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine hydrochloride, phenol, piperocaine, piridocaine, polidocanol, pramoxine, prilocalne, procaine, propanocaine, proparacaine, propipocaine, propoxycaine hydrochloride, pseudococaine, pyrrocaine, ropavacaine, salicyl alcohol, tetracaine hydrochloride, tolycaine, trimecaine, and zolamine, or pharmaceutically acceptable salts thereof, or mixtures of two or more of these.

Other preferred therapeutic agents include vasodilators, referred to herein as vasospasm-inhibiting agents, such as a calcium channel blocker or a nitrate, which suppress vasospasm. Vasospasm is a common complication in patients following angioplasty procedures. Vasospasm occurs as a response to injury of a blood vessel, and the tendency toward vasospasm decreases as the vessel heals. Accordingly, the vasospasm-inhibiting agent is desirably supplied over a period of about two to three weeks. Of course, trauma from angioplasty is not the only vessel injury that can cause vasospasm, and the medical device 10 may be introduced into vessels other than the coronary arteries, such as, without limitation, the aorta, carotid arteries, renal arteries, iliac arteries or peripheral arteries for the prevention of vasospasm at those locations.

A particularly useful vasospasm-inhibiting agent can be selected, without limitation, from the group consisting of alibendol, ambucetamide, aminopromazine, apoatropine, bevonium methyl sulfate, bietamiverine, butaverine, butropium bromide, n-butylscopolammonium bromide, caroverine, cimetropium bromide, cinnamedrine, clebopride, coniine hydrobromide, coniine hydrochloride, cyclonium iodide, difemerine, diisopromine, dioxaphetyl butyrate, diponium bromide, drofenine, emepronium bromide, ethaverine, feclemine, fenalamide, fenoverine, fenpiprane, fenpiverinium bromide, fentonium bromide, flavoxate, flopropione, gluconic acid, guaiactamine, hydramitrazine, hymecromone, leiopyrrole, mebeverine, moxaverine, nafiverine, octamylamine, octaverine, oxybutynin chloride, pentapiperide, phenamacide hydrochloride, phloroglucinol, pinaverium bromide, piperilate, pipoxolan hydrochloride, pramiverin, prifinium bromide, properidine, propivane, propyromazine, prozapine, racefemine, rociverine, spasmolytol, stilonium iodide, sultroponium, tiemonium iodide, tiquizium bromide, tiropramide, trepibutone, tricromyl, trifolium, trimebutine, n,n-1trimethyl-3,3-diphenyl-propylamine, tropenzile, trospium chloride, and xenytropium bromide, or pharmaceutically acceptable salts thereof, or mixtures of two or more of these.

Especially useful therapeutic agents employed in the present invention include thrombolytics and antithrombogenics irrespective of the sort of medical device to which these therapeutic agents are attached. Utility of these therapeutic agents is particularly noted when the structure 12 is a vascular stent. Thrombolytics are therapeutic agents that dissolve, break up or disperse thrombi, which are blood clots found in blood vessels at the site where they formed. Antithrombogenics are therapeutic agents that interfere with or prevent the formation of thrombi.

Particularly preferred thrombolytic agents include, without limitation, urokinase, streptokinase, and a tissue plasminogen activator, or a mixture of two or more of these. Urokinase is a plasminogen activating enzyme typically obtained from human kidney cell cultures. Urokinase catalyzes the conversion of plasminogen into the fibrinolytic plasmin, which breaks down fibrin thrombi.

Particularly preferred antithrombogenic agents include, without limitation, an antiplatelet, argatroban, aspirin, heparin, covalent heparin, a glycoprotein Ilb/lila inhibitor, hirudin, hirulog, D-phenylalanyl-L-poly-L-arginyl chloromethyl ketone, and ticlopidine, or a mixture of two or more of these. Heparin is a mucopolysaccharide anticoagulant typically obtained from porcine intestinal mucosa or bovine lung. Heparin acts as a thrombin inhibitor by greatly enhancing the effects of the blood's endogenous antithrombin III. Thrombin, a potent enzyme in the coagulation cascade, is key in catalyzing the formation of fibrin. Therefore, by inhibiting thrombin, heparin inhibits the formation of fibrin thrombi.

An antiproliferative agent such as methotrexate will inhibit over-proliferation of smooth muscle cells and thus inhibit restenosis of a dilated segment of a blood vessel. The antiproliferative agent is desirably supplied for this purpose over a period of about four to six months. Additionally, localized delivery of an antiproliferative agent is also useful for the treatment of a variety of malignant conditions characterized by highly vascular growth. In such cases, the medical device 10 of the present invention could be placed in the arterial supply of the tumor to provide a means of delivering a relatively high and prolonged dose of the antiproliferative agent directly to the tumor, while limiting systemic exposure and toxicity. Indeed, the antiproliferative agent so employed may be a curative, a pre-operative debulker reducing the size of the tumor, or a palliative that eases the symptoms of the cancer.

Accordingly, preferred therapeutic agents used in the context of the present invention include antiproliferative agents that are an antimitotic agent, a microtubule inhibitor, an actin inhibitor, a growth hormone antagonist, a growth factor or an anti-growth factor antibody, or a growth factor antagonist, including a PDGF antagonist. More particularly, the preferred therapeutic agents having antiproliferative activity include, without limitation, angiogenin, angiopeptin, betaxolol hydrochloride, colchicine, cytochalasin, finasteride, methotrexate, paclitaxel, tamoxifen citrate, trapidal, docetaxel, betulinic acid, etoposide, podophyllotoxin, tetrahydrocannabinol, vindesine, epothilone A, epothilone B, epothilone C, epothilone D, epothilone E, epothilone F, ixabepilone, camptothecin, colchicine, topotecan, vinblastine, vincristine, and vindesine, and analogs thereof, or pharmaceutically acceptable salts thereof, or mixtures of two or more of these.

Imparting therapeutic agents that impact blood flow can be particularly important in patients having cardiovascular disease. Accordingly, therapeutic agents in the category of the vasodilating agent can be highly valued. Suitable ones usefully employed in the context of the present invention include, without limitation, those selected from the group consisting of a calcium channel blocker, a nitrate, nitric oxide, and a nitric oxide promoter, or a mixture of two or more of these.

Another category of therapeutic agents also used for cardiovascular patients is the antihypertensive agent, which include, without limitation, α-1-adrenergic blockers, beta blockers, angiotensin-converting enzyme (ACE) inhibitors, angiotensin-II-antagonists, and calcium channel blockers. Preferred alpha-1-adrenergic blockers include, without limitation, doxazosine, indoramine, ketanserine, prazosine, terazosine, trimazosine, and urapidil. Preferred beta blockers include, without limitation, atenolol, bisoprolol, metoprolol, nadolol, propranolol, tenoretic, and timolol. Preferred ACE inhibitors include, without limitation, benazepril, captopril, enalapril, lisinopril, moexipril, and trandolapril. Preferred angiotensin-II antagonists include, without limitation, candesartan, eprosartan, irbesartan, losartan, telmisartan, and valsartan. Preferred calcium channel blockers, both with respect to the antihypertensive agent and the vasodilating agent, and without limitation, include amlodipine, diltiazem, felodipine, and verapamil.

Another important category of therapeutic agents usefully employed with the present invention is the antisecretory agent, which is preferably dimethyl sulfoxide or a retinoid, or a mixture thereof.

Therapeutic agents having immunosuppressive effect in patients are important in the context of transplant surgery and autoimmune diseases, among others. Accordingly, preferred immunosuppressive agents are included for use in the context of the present invention, and are selected from the group consisting of cyclosporine, ciclosporin, tacrolimus, sirolimus, mycophenolate mofetil, OKT3, thymoglobulin, daclizumab, basiliximab, and ISA247.

Another embodiment of the present invention employs as the therapeutic agent dopamine or a dopamine agonist such as bromocriptine mesylate or pergolide mesylate, or a mixture of two or more of these. Such therapeutic agents are useful for the treatment of neurological disorders such as Parkinson's disease. The medical device 10 can be placed in the vascular supply of the thalamic substantia nigra for this purpose, or elsewhere, localizing treatment in the thalamus.

For purposes of radiotherapy, another valuable category of therapeutic agents are radiotherapeutic agents, which are isotopes selected from the group consisting of ⁶⁰Co, ¹⁹²Ir, ³²p, ¹¹¹In, ⁹⁰Y, and ^{99m}Tc. Another category of radioactive therapeutic agents are any of the therapeutic agents radiolabeled with one or more isotopes selected from the group consisting of ¹⁴C, ³H, ¹³¹I, ³²P, and ³⁶S.

A form of prophylactic as well as ameliorative treatment involves the complexing and removal of oxidizing species, other free radicals, and excess iron, as are known in the art. Accordingly, another category of therapeutic agents is antioxidants, including, for example, ascorbic acid, α-tocopherol, and a 21-aminosteroid (lasaroid). Free radical scavengers represent yet another valuable category of therapeutic agents, such as, for example, superoxide dismutase. For the removal of excess iron in a patient's blood, the category of therapeutic agents known as iron chelators is important, such as, for example, deferoxamine.

When the structure 12 is configured as a vascular stent, particularly preferred materials for the therapeutic agents of the layer 18 include a thrombolytic agent, such as heparin, anti-inflammatory steroids including, but not limited to, dexamethasone and its derivatives, and mixtures of preferably heparin and such steroids.

Description Regarding Medical Device Design and Coating

With reference to FIG. 1 or 2, the medical device 10 in one embodiment of the present invention also comprises at least one protecting layer 20 positioned over the layer 18 of therapeutic agent. The purpose of the protecting layer 20 is to provide a controlled release of the therapeutic agent when the medical device 10 is positioned in the vascular system of a patient. The thickness of the protecting layer 20 is chosen so as to provide such control. In addition to functioning to control the release of the therapeutic agent of layer 18, the protecting layer 20 also protects the therapeutic agent of layer 18 during deployment of the medical device 10, for example, during insertion of the medical device 10 through a catheter and into the vascular system or elsewhere in the patient.

More particularly, the protecting layer 20 is composed of a polymer deposited on the therapeutic agent layer 18 by the well-known process of vapor deposition. Plasma deposition is also useful for this purpose. Preferably, the layer 20 is one that is polymerized from a vapor that is free of any solvent, catalysts or similar polymerization promoters. Also preferably, the polymer in the protecting layer 20 is one that automatically polymerizes upon condensation from the vapor phase, without the action of any curative agent or activity such as heating, the application of visible or ultraviolet light, radiation, ultrasound or the like. Optimally, the polymer in the protecting layer 20 is polyimide, parylene or a parylene derivative, as set forth in U.S. Pat. No. 5,609,629, which is incorporated herein by reference.

As indicated in the embodiment shown in FIG. 2, the medical device 10 of the present invention can further comprise at least one primer layer 16 positioned between the material 14 of structure 12 and the at least one layer 18 of the therapeutic agent. While the primer layer 16 can be a medical grade primer, as is known in the art, the function of which is to lessen the physiological impact of the material 14 and/or increase the amount of or stabilize the therapeutic agent of layer 18, relative to the untreated material 14, the primer layer 16 can also be composed of the same polymer as the at least one protecting layer 20. However, the primer layer 16 is also preferably less porous than the at least one protecting layer 20, and is more preferably substantially nonporous. “Substantially nonporous” means that the primer layer 16 is sufficiently impervious to prevent any appreciable interaction between the material 14 of the structure 12 and the blood to which the medical device 10 will be exposed during use. The use of a primer layer 16 that is substantially nonporous would permit the use of a toxic or poisonous material 14, as mentioned above. Even if the material 14 of the structure 12 is biocompatible, however, it may be advantageous to isolate it from the blood by use of a substantially nonporous primer layer 16.

When the at least one layer 18 of therapeutic agent contains a relatively soluble material such as heparin, the at least one layer 18 preferably contains a total of about 1 to about 4 mg of therapeutic agent per cm² of the gross surface area of the structure 12. This provides a release rate for the heparin (measured in vitro) that is desirably in the range of about 0.1 to about 0.5 mg/cm² per day, and preferably about 0.25 mg/cm² per day, under typical blood flows through vascular stents, for example. It should be noted that the solubility of dexamethasone can be adjusted as desired, with or without the inclusion of heparin, by mixing it with one or more of its relatively more soluble derivatives, such as dexamethasone sodium phosphate.

As shown in FIG. 3, the medical device 10 of the present invention is not limited to the inclusion of a coating having a single layer 18 of therapeutic agent. The medical device 10 can, for example, have a coating that comprises a second layer 22 of a second therapeutic agent positioned over the structure 12, immediately on top of the therapeutic agent of layer 18. The second therapeutic agent of the second layer 22 can be, but need not necessarily be, different from the therapeutic agent of the first therapeutic agent layer 18. The use of different materials in the layers 18 and 22 allows the medical device 10 to perform more than a single therapeutic function, or perform a single therapeutic function more effectively.

The medical device 10 of the present invention can further comprise an additional protecting layer 24 of the polymer positioned between each of the layers 18 and 22 of therapeutic agent, as shown in FIG. 4. The additional protecting layer 24 can give the therapeutic agents in the layers 18 and 22 different release rates. Simultaneously, or alternatively, the medical device 10 can employ therapeutic agents in the two layers 18 and 22 that are different from one another and have differing solubilities. In such a case, it is advantageous and preferred to position the layer 22 containing the less soluble therapeutic agent above the layer 18 containing the more soluble therapeutic agent.

For example, when the structure 12 of the medical device 10 is configured as a vascular stent, it is advantageous for the at least one layer 18 to contain relatively soluble heparin, and the second layer 22 to contain relatively less soluble dexamethasone. Unexpectedly, the heparin promotes the release of the dexamethasone, increasing its release rate many times over the release rate of dexamethasone in the absence of heparin. The release rate of the heparin is also lowered, somewhat less dramatically than the increase of the dexamethasone release rate. When a layer 22 of dexamethasone is disposed over a layer 18 of heparin, and beneath a porous parylene protecting layer 20, as set forth in detail in the aforementioned U.S. Pat. No. 5,609,629, the dexamethasone can release at a desirable rate of from about 1 to about 10 μg/cm² per day.

The therapeutic agent layers 18 and/or 22 are preferably applied to the device 10 independent of the application of the protecting layers 20 and/or 24. Any mixing of a therapeutic agent from the layers 18 and/or 20 into the protecting layers 20 and/or 24, prior to introducing the device 10 into the patient, is unintentional and merely incidental. This gives significantly more control over the release rate of the therapeutic agent than the simple dispersal of a therapeutic agent in a polymeric layer.

The medical device 10 may not need to include the additional protecting layer 24 when two or more layers 18 and 22 of therapeutic agent are present. As shown in FIG. 3, the layers 18 and 22 do not have to be separated by a layer, but can instead lie directly against one another. And should additional amounts of a given therapeutic agent be advantageous relative to what is loaded per a single application of layer 18 or 22, for example, separation of additional layers of the same therapeutic agent may or may not be separated by a layer designed, for example, to allow sequential release of the same therapeutic agent, thereby allowing for an extended elution profile of a therapeutic agent. It is still advantageous in this embodiment to position the layer 22 containing the relatively less soluble therapeutic agent above the layer 18 containing the relatively more soluble therapeutic agent.

The particular design of the therapeutic agents attached to the medical device 10 as disclosed can be adapted to specific uses in a variety of ways. For example, the medical device 10 may include further layers of the same or different therapeutic agents. These additional layers of therapeutic agent may or may not be separated by additional layers, as convenient or desired. Alternatively, additional protecting layers may separate only some of the additional layers of therapeutic agent. Moreover, one therapeutic agent can be placed on one portion of the structure 12 of the medical device 10, and another therapeutic agent placed on a different portion of the structure 12 of the medical device 10. Such spatial differentiation can be achieved using many methods well-known in the art, including masking the implantable device on area(s) where a particular therapeutic agent is not desired prior to its deposition (via spraying, vaporization, and the like, as appropriate), followed by removal of the mask. For those embodiments that incorporate a second therapeutic agent, the implantable structure can be masked again or not, as appropriate.

In one embodiment, the medical device 10 does not include the primer layer 16. Such a configuration is shown in FIG. 1, in which the therapeutic agent layer 18 is positioned directly atop the material 14 of the structure 12. In such a case, it may be highly advantageous to surface process or surface activate the material 14, to promote the deposition or adhesion of the therapeutic agent on the material 14, especially before the positioning of the at least one protecting layer 20. Surface processing and surface activation can also selectively alter the release rate of the therapeutic agent. Such processing can also be used to promote the deposition or adhesion of the primer layer 16, as shown in FIGS. 2-4, on the material 14. The primer layer 16 itself, or any second or additional protecting layer 24 itself, can similarly be processed to promote the deposition or adhesion of the therapeutic agent layer 18, or to further control the release rate of the therapeutic agent.

Useful methods of surface processing can include any of a variety of well-known procedures, including: cleaning; physical modifications such as etching or abrasion; and chemical modifications such as solvent treatment, the application of primer coatings, the application of surfactants, plasma treatment, ion bombardment and covalent bonding.

For deposition of hydrophilic therapeutic agents especially, it has been found particularly advantageous to plasma treat the additional primer layer 16 (for example, of parylene) before depositing the therapeutic agent layer 18 atop it. The plasma treatment improves the adhesion of, increases the amount of, and allows the hydrophilic therapeutic agent to be deposited in a more uniform layer. Indeed, it is very difficult to deposit a hygroscopic agent such as heparin on an unmodified hydrophobic and thus poorly wettable surface, such as that provided by parylene. However, plasma treatment renders a parylene surface wettable, allowing heparin, for example, to be easily deposited on it.

In contrast, for deposition of hydrophobic therapeutic agents, it is advantageous to use a material for layer 16 that is itself hydrophobic, such as parylene, before depositing the therapeutic agent layer 18 atop it. For example, a medical device 10 having paclitaxel or docetaxel deposited thereon is a particularly preferred embodiment of the present invention. In addition to use of sugar as disclosed elsewhere herein, choices made for the primer 16 of the therapeutic agent layer 18 can impact both the quantity of therapeutic agent that is deposited and the rate at which it elutes from the medical device 10.

Any of the porous polymer protecting layers 20 and 24 may also be surface processed by any of the methods mentioned above to alter the amount and/or release rate of the therapeutic agent applied thereon, and/or otherwise improve the biocompatibility of the surface of the protecting layers. For example, the application of an overcoat of polyethylene oxide, phosphatidylcholine or covalently attached heparin to the protecting layers 20 and/or 24 could render the surface of the layers more blood compatible. Similarly, the plasma treatment or application of a hydrogel coating to the protecting layers 20 and/or 24 could alter their surface energies, preferably providing surface energies in the range of 20 to 30 dyne/cm, thereby rendering their surfaces more biocompatible.

Referring now to FIG. 5, an embodiment of the medical device 10 is shown in which a mechanical bond or connector 26 is provided between (a) any one of the protecting layers 20 and 24, and (b) any or all of the other of the protecting layers 20 and 24, the primer layer 16 and the material 14. The connector 26 reliably secures the layers 16, 20 and/or 24 to each other, and/or to the material 14. The connector 26 lends structural integrity to the medical device 10, particularly after the therapeutic agent layer or layers 18 and/or 20 have been fully released into the patient.

For simplicity, the connector 26 is shown in FIG. 5 as a plurality of projections of the material 14 securing a single protecting layer 20 to the material 14. The connector 26 may alternatively extend from the protecting layer 20, through the therapeutic agent layer 18, and to the material 14. In either case, a single layer 18 of therapeutic agent, divided into several segments by the connector 26, is positioned between the protecting layer 20 and the material 14. The connectors can also function to partition the different therapeutic agents into different regions of the device's surface.

The connector 26 can, of course, be provided in a variety of ways. For example, the connector 26 can be formed as a single piece with the material 14 during its initial fabrication or molding into the structure 12. The connector 26 can instead be formed as a distinct element, such as a bridge, strut, pin or stud added to an existing structure 12. The connector 26 can also be formed as a built-up land, shoulder, plateau, pod or pan on the material 14. Alternatively, a portion of the material 14 between the desired locations of plural connectors 26 can be removed by etching, mechanical abrasion or the like, and the therapeutic agent layer 18 deposited between them. The connector 26 can also be formed so as to extend downwards towards the material 14, by wiping or etching away a portion of a previously applied therapeutic agent layer 18, and allowing the protecting layer 20 to deposit by vapor deposition or plasma deposition directly on the bare portions of the material 14. Other ways to expose a portion of the material 14 to direct connection to the protecting layer 20 should be evident to those skilled in this area.

The other details of the construction or composition of the various elements of the disclosed embodiments of the present invention are not believed to be critical to the achievement of the advantages of the present invention, so long as the elements possess the strength or flexibility needed for them to perform as disclosed. In view of the present disclosure, the selection of these and other details of construction are believed to be well within the ability of one of even rudimentary skills in this area.

The above-described medical device is merely an illustrative embodiment of the principles of this invention, and that other devices and methods for using and making them may be devised by those skilled in the art without departing from the spirit and scope of the invention. The invention is directed at embodiments both comprising and consisting of the disclosed parts. It is contemplated that only part of a device need be coated, although some embodiments may entail complete coating in accordance with the principles set forth hereinabove. Furthermore, different parts of the device can be coated with different therapeutic agents or protecting layers. It is also contemplated that different sides or regions of the same part of a device can be coated with different therapeutic agents or protecting layers.

We claim:

1. A medical device comprising:
(a) an implantable structure having a surface,
(b) at least one layer posited on at least a portion of the surface, the layer comprising a mono- or disaccharide sugar and at least one therapeutic agent.

2. The medical device of claim 1, wherein the sugar is present in the layer in an amount that is from about 10% to about 25% of the weight of the therapeutic agent.

3. The medical device of claim 2, wherein the surface comprises at least two portions, the first portion having a first concentration of sugar and the second portion having a second, greater concentration of sugar.

4. The medical device of claim 3, wherein the layer is contiguous over the first and second portions.

5. The medical device of claim 3, wherein the layer is non-contiguous over the first and second portions.

6. The medical device of claim 1, wherein the sugar is selected from the group consisting of galactose, glucose, mannose, xylose, sucrose, and trehalose.

7. The medical device of claim 1, wherein the implantable structure is selected from the group consisting of a stent, a wire guide, a catheter, a monitor, a prosthesis, a cannula, a graft, a cardiac pacemaker lead, a cardiac defibrillator lead, a suture, a needle, an angioplasty device, a pacemaker, an orthopedic device, appliance, implant or replacement, a scalpel, a scissors, a forceps, and a portion of any of these.

8. The medical device of claim 1, wherein the surface comprises a material selected from the group consisting of a biocompatible metal, carbon or carbon fiber, a biocompatible polymer, a biodegradable polymer, and an extracellular matrix component.

9. The medical device of claim 8, wherein the biocompatible metal is selected from the group consisting of stainless steel, tantalum, titanium, nitinol, gold, platinum, inconel, iridium, silver, and tungsten, or alloys of any of these.

10. The medical device of claim 1, wherein the therapeutic agent is selected from the group consisting of an anti-inflammatory agent, an analgesic agent, a local anesthetic agent, a vasospasm-inhibiting agent, a thrombolytic agent, an antithrombogenic agent, an antiproliferative agent, a fibrinolytic agent, a vasodilating agent, an antihypertensive agent, an antimicrobial agent, an antifungal agent, an antisecretory agent, an immunosuppressive agent, a dopamine agonist, a radiotherapeutic agent, a biological agent, an angiotensin converting enzyme (ACE) inhibitor, an antioxidant, a free-radical scavenger, and an iron chelator, or a radiolabelled form thereof, or mixtures of two or more of these.

11. The medical device of claim 10, wherein the therapeutic agent is the antiproliferative agent.

12. The medical device of claim 11, wherein the antiproliferative agent inhibits microtubule disassembly or mitosis.

13. The medical device of claim 1, wherein the therapeutic agent is selected from the group consisting of paclitaxel, docetaxel, epothilone A, epothilone B, epothilone C, epothilone D, epothilone E, epothilone F, ixabepilone, camptothecin, colchicine, topotecan, vinblastine, vincristine, and vindesine, and analogs thereof.

14. The medical device of claim 1, wherein the layer comprises at least two different therapeutic agents.

15. The medical device of claim 1, wherein the surface comprises at least two portions, the first portion having a first therapeutic agent and the second portion having a second, different therapeutic agent.

16. The medical device of claim 1, further comprising a second layer posited on at least a portion of the at least one layer, the second layer comprising a mono- or disaccharide sugar and at least one therapeutic agent.

17. A method of delivering a therapeutic agent, comprising implanting a medical device comprising a surface and at least one layer posited on at least a portion of the surface, the layer comprising at least one mono- or disaccharide sugar and at least one therapeutic agent.

18. The method of claim 17, wherein the surface comprises (i) a first portion that contains a first concentration of sugar and a first concentration of therapeutic agent and (ii) a second portion that contains a second concentration of sugar and a second concentration of therapeutic agent both of which concentrations are higher than those in the first portion.

19. The method of claim 17, wherein a second layer is posited on at least a portion of the first layer, the second layer comprising at least one mono- or disaccharaide sugar and at least one therapeutic agent different from the at least one therapeutic agent in the first layer.

20. A method for positing a therapeutic agent on a medical device comprising forming a composition of the therapeutic agent and a mono- or disaccharide sugar, contacting at least a part of the medical device with the composition for a time sufficient to allow the composition to adhere to the medical device.
